(19) United States
(12) Patent Application Publication
(10) Pub. No.: US 2024/0160998 A1
(43) Pub. Date: May 16, 2024

(54) REPRESENTING ATOMIC STRUCTURES AS A GAUSSIAN PROCESS

(71) Applicant: TOYOTA RESEARCH INSTITUTE, INC., Los Altos, CA (US)

(72) Inventors: Jens Strabo HUMMELSHØJ, Millbrae, CA (US); Joseph Harold MONTOYA, Millbrae, CA (US)

(73) Assignees: TOYOTA RESEARCH INSTITUTE, INC., Los Altos, CA (US); TOYOTA JIDOSHA KABUSHIKI KAISHA, Aichi-Ken (JP)

(21) Appl. No.: 17/987,530

(22) Filed: Nov. 15, 2022

(51) Int. Cl. G06N 20/00

(52) U.S. Cl. CPC G06N 20/00

(57) ABSTRACT

A method for representing atomic structures as Gaussian processes is described. The method includes mapping a crystal structure of chemical elements in a real space, in which atoms of the chemical elements are represented in a unit cell. The method also includes learning, by a machine learning model, a 3D embedding of each of the chemical elements in the real space according to the mapping of the crystal structure of the chemical elements. The method further includes training the machine learning model according to a representation of the atoms of the chemical elements in the unit cell based on the mapping of the crystal structure of the chemical elements. The method also includes predicting a material property corresponding to a point within the real space.

### TECHNICAL FIELD

Certain aspects of the present disclosure generally relate to artificial neural networks and, more particularly, to representing atomic structures as Gaussian processes.

### BACKGROUND

An artificial neural network, which may include an interconnected group of artificial neurons, may be a computational device or may represent a method to be performed by a computational device. Artificial neural networks may have corresponding structure and/or function in biological neural networks. Artificial neural networks, however, may provide useful computational techniques for certain applications, in which traditional computational techniques may be cumbersome, impractical, or inadequate. Because artificial neural networks may infer a function from observations, such networks may be useful in applications where the complexity of the task and/or data makes the design of the function burdensome using conventional techniques.

Machine learning may be used to perform both materials discovery and predict properties of the materials faster than molecular simulations. Machine learning can help identify correlations between material features and target properties. Nevertheless, it is challenging to represent an atomic structure in an understandable format. Conventionally, representations of an atomic structure generally rely on a graph representation. Techniques for representing atomic structures as a Gaussian process are desired.

### SUMMARY

A method for representing atomic structures as Gaussian processes is described. The method includes mapping a crystal structure of chemical elements in a real space, in which atoms of the chemical elements are represented in a unit cell. The method also includes learning, by a machine learning model, a 3D embedding of each of the chemical elements in the real space according to the mapping of the crystal structure of the chemical elements. The method further includes training the machine learning model according to a representation of the atoms of the chemical elements in the unit cell based on the mapping of the crystal structure of the chemical elements. The method also includes predicting a material property corresponding to a point within the real space.

A non-transitory computer-readable medium having program code recorded thereon for representing atomic structures as Gaussian processes is described. The program code is executed by a processor. The non-transitory computer-readable medium includes program code to map a crystal structure of chemical elements in a real space, in which atoms of the chemical elements are represented in a unit cell. The non-transitory computer-readable medium also includes program code to learn, by a machine learning model, a 3D embedding of each of the chemical elements in the real space according to the mapping of the crystal structure of the chemical elements. The non-transitory computer-readable medium further includes program code to train the machine learning model according to a representation of the atoms of the chemical elements in the unit cell based on the mapping of the crystal structure of the chemical elements. The non-transitory computer-readable medium also includes program code to predict a material property corresponding to a point within the real space.

A system for representing atomic structures as Gaussian processes is described. The system includes a neural processing unit (NPU), and a memory coupled to the NPU. The instructions stored in the memory and operable, when executed by the NPU, cause the system to map a crystal structure of chemical elements in a real space, in which atoms of the chemical elements are represented in a unit cell. The instructions further cause the system to learn, by a machine learning model, a 3D embedding of each of the chemical elements in the real space according to the mapping of the crystal structure of the chemical elements. The instructions also cause the system to train the machine learning model according to a representation of the atoms of the chemical elements in the unit cell based on the mapping of the crystal structure of the chemical elements. The instructions further cause the system to predict a material property corresponding to a point within the real space.

This has outlined, rather broadly, the features and technical advantages of the present disclosure in order that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

The features, nature, and advantages of the present disclosure will become more apparent from the detailed description set forth below when taken in conjunction with the drawings in which like reference characters identify correspondingly throughout.

FIG. 1 illustrates an example implementation of designing a neural network using a system-on-chip (SoC), including a general purpose processor, in accordance with certain aspects of the present disclosure.

FIGS. 2A, 2B, and 2C are diagrams illustrating a neural network, in accordance with aspects of the present disclosure.

FIG. 2D is a diagram illustrating a neural network, in accordance with aspects of the present disclosure.

FIG. 3 is a block diagram illustrating a unit cell containing a hydrogen and a lithium atom and their associated three dimensional (3D) embeddings, in accordance with aspects of the present disclosure.

FIG. 4 illustrates a two dimensional (2D) heatmap showing the Gaussian process (GP) interpolations of a single dimension for a 2D hexagonal lattice with two atoms in the unit cell, according to aspects of the present disclosure.

FIG. 5 illustrates a 3D plot showing a bravais lattice of an atom using a face centered cubic (FCC) covariance function, according to aspects of the present disclosure.

FIG. 6 is a flow diagram illustrating a method for representing atomic structures as Gaussian processes, according to aspects of the present disclosure.

### DETAILED DESCRIPTION

The detailed description set forth below, in connection with the appended drawings, is intended as a description of various configurations and is not intended to represent the only configurations in which the concepts described may be practiced. The detailed description includes specific details for the purpose of providing a thorough understanding of the various concepts. Nevertheless, it will be apparent to those skilled in the art that these concepts may be practiced without these specific details. In some instances, well-known structures and components are shown in block diagram form in order to avoid obscuring such concepts.

Based on the teachings, one skilled in the art should appreciate that the scope of the disclosure is intended to cover any aspect of the disclosure, whether implemented independently of or combined with any other aspect of the disclosure. For example, an apparatus may be implemented or a method may be practiced using any number of the aspects set forth. In addition, the scope of the disclosure is intended to cover such an apparatus or method practiced using other structure, functionality, or structure and functionality in addition to or other than the various aspects of the disclosure set forth. It should be understood that any aspect of the disclosure disclosed may be embodied by one or more elements of a claim.

Although particular aspects are described, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different technologies, system configurations, networks, and protocols, some of which are illustrated by way of example in the figures and in the following description of the preferred aspects. The detailed description and drawings are merely illustrative of the disclosure, rather than limiting the scope of the disclosure being defined by the appended claims and equivalents thereof.

Machine learning is concerned with the automatic discovery of patterns in data through the use of computer algorithms. Once discovered, these patterns may be used to perform data classification and/or value prediction. With growing experimental and simulated dataset size for materials science research, the ability of algorithms to automatically learn and improve from data becomes increasingly useful. Various types of machine learning algorithms, such as neural networks, have recently been applied to materials research. Among these machine learning algorithms, convolutional neural networks (CNNs) have been very attractive in recent years due to their great success in image recognition.

A CNN may be composed of multilayer neural networks, of which at least one layer employs a mathematical operation called “convolution” to enable the CNN to extract high-level features directly from data. Compared to many other algorithms that specify artificial features based on domain knowledge, a CNN involves relatively little pre-processing, as the features can be directly learned from the data. This is particularly useful when the features are difficult to exactly define. Unlike their long-used basic forms, such as perceptron and fully connected neural networks, CNNs are very recently used for solving solid state problems, such as learning material property prediction, material classification, and material phase transition identification.

Another advantage of neural networks is that they are easy to utilize in transfer learning, which means that a neural network first learns from a large database with inexpensive labels (e.g., first principles calculation results), and then it is fine-tuned on a small dataset where much fewer labeled samples are available (e.g., experimental data). This technique can be used to overcome the data scarcity problem in materials research, and it has been applied to property prediction of small molecules and crystalline compounds very recently.

The practical realization and sustainable future of emergent technologies is dependent on accelerating materials discovery. Data-driven methods are anticipated to play an increasingly significant role in enabling this desired acceleration. While the vision of accelerating materials discovery using data-driven methods is well-founded, practical realization is throttled due to challenges in data generation, ingestion, and materials state-aware machine learning. High-throughput experiments and automated computational workflows are addressing the challenge of data generation, and capitalizing on these emerging data resources involves ingestion of data into an architecture that captures the complex provenance of experiments and simulations.

In practice, machine learning may be used to perform both materials discovery and predict properties of the materials faster than molecular simulations. Machine learning can help identify correlations between material features and target properties. Unfortunately, representing an atomic structure in an understandable format is challenging. Some prior art methodologies represent an atomic structure using a graph representation, rather than representing the atomic structure of a material as a Gaussian process. Continued material discovery using machine learning models involves an improved material representation.

Some aspects of the present disclosure are directed to representing a crystal structure of a material using a Gaussian process. In some aspects of the present disclosure, the Gaussian process represents the crystal structure of the material using individual atoms of a unit cell that serve as training vectors, such that the crystal structure is mapped to a real space. Following the mapping of the crystal structure to the real space, each point in the real space can be learned, such that properties (e.g., energy density and electron density) and a high dimensional representation of chemical elements of the material can be learned and predicted.

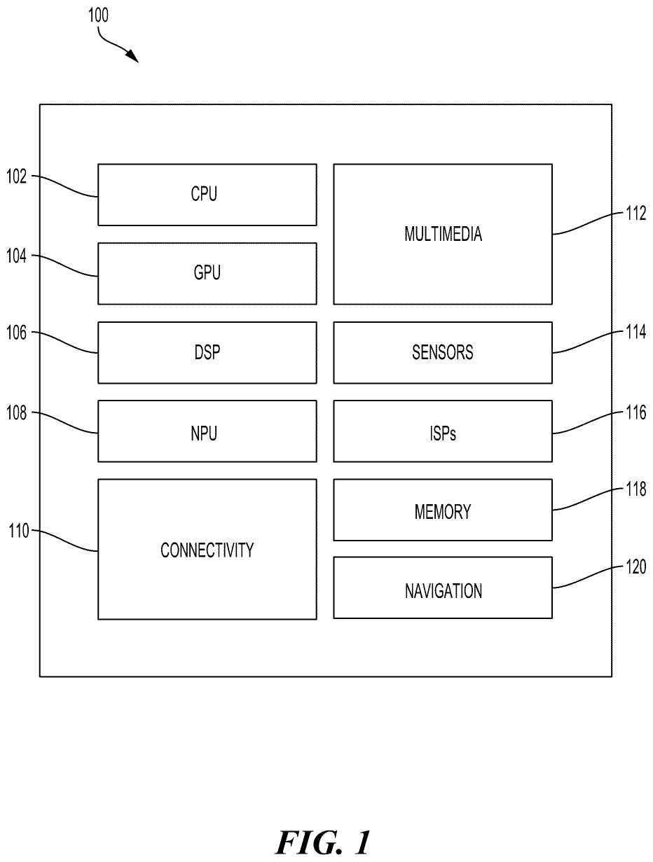

FIG. 1 illustrates an example implementation of a system-on-chip (SoC) 100, which may include a central processing unit (CPU) 102 or multi-core CPUs, in accordance with certain aspects of the present disclosure, such an artificial intelligence (AI) accelerator. Variables (e.g., neural signals and synaptic weights), system parameters associated with a computational device (e.g., neural network with weights), delays, frequency bin information, and task information may be stored in a memory block associated with a neural processing unit (NPU) 108, in a memory block associated with a CPU 102, in a memory block associated with a graphics processing unit (GPU) 104, in a memory block associated with a digital signal processor (DSP) 106, in a memory block 118, or may be distributed across multiple blocks. Instructions executed at the CPU 102 may be loaded from a program memory associated with the CPU 102 or may be loaded from a memory block 118.

The SoC 100 may also include additional processing blocks tailored to specific functions, such as a connectivity block 110, which may include fifth generation (5G) new radio (NR) connectivity, fourth generation long term evolution (4G LTE) connectivity, unlicensed Wi-Fi connectivity, USB connectivity, Bluetooth connectivity, and the like, and a multimedia processor 112 that may, for example, detect and recognize gestures. In one implementation, the NPU is implemented in the CPU, DSP, and/or GPU. The SoC 100 may also include a sensor processor 114 to provide sensor image data, image signal processors (ISPs) 116, and/or navigation module 120, which may include a global positioning system.

Deep learning architectures may perform an object recognition task by learning to represent inputs at successively higher levels of abstraction in each layer, thereby building up a useful feature representation of the input data. In this way, deep learning addresses a major bottleneck of traditional machine learning. Prior to the advent of deep learning, a machine learning approach to an object recognition problem may have relied heavily on human engineered features, perhaps in combination with a shallow classifier. A shallow classifier may be a two-class linear classifier, for example, in which a weighted sum of the feature vector components may be compared with a threshold to predict to which class the input belongs. Human engineered features may be templates or kernels tailored to a specific problem domain by engineers with domain expertise. Deep learning architectures, in contrast, may learn to represent features that are similar to what a human engineer might design, but through training. Furthermore, a deep network may learn to represent and recognize new types of features that a human might not have considered.

A deep learning architecture may learn a hierarchy of features. If presented with visual data, for example, the first layer may learn to recognize relatively simple features, such as edges, in the input stream. In another example, if presented with auditory data, the first layer may learn to recognize spectral power in specific frequencies. The second layer, taking the output of the first layer as input, may learn to recognize combinations of features, such as simple shapes for visual data or combinations of sounds for auditory data. For instance, higher layers may learn to represent complex shapes in visual data or words in auditory data. Still higher layers may learn to recognize common visual objects or spoken phrases.

Deep learning architectures may perform especially well when applied to problems that have a natural hierarchical structure. For example, the classification of motorized vehicles may benefit from first learning to recognize wheels, windshields, and other features. These features may be combined at higher layers in different ways to recognize cars, trucks, and airplanes.

Neural networks may be designed with a variety of connectivity patterns. In feed-forward networks, information is passed from lower to higher layers, with each neuron in a given layer communicating to neurons in higher layers. A hierarchical representation may be built up in successive layers of a feed-forward network, as described above. Neural networks may also have recurrent or feedback (also called top-down) connections. In a recurrent connection, the output from a neuron in a given layer may be communicated to another neuron in the same layer. A recurrent architecture may be helpful in recognizing patterns that span more than one of the input data chunks that are delivered to the neural network in a sequence. A connection from a neuron in a given layer to a neuron in a lower layer is called a feedback (or top-down) connection. A network with many feedback connections may be helpful when the recognition of a high-level concept may aid in discriminating the particular low-level features of an input.

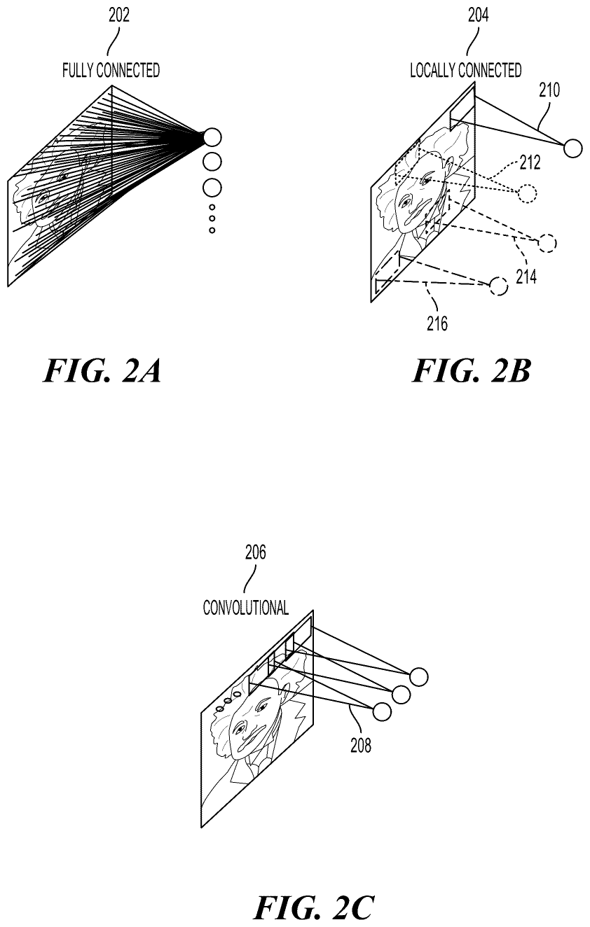

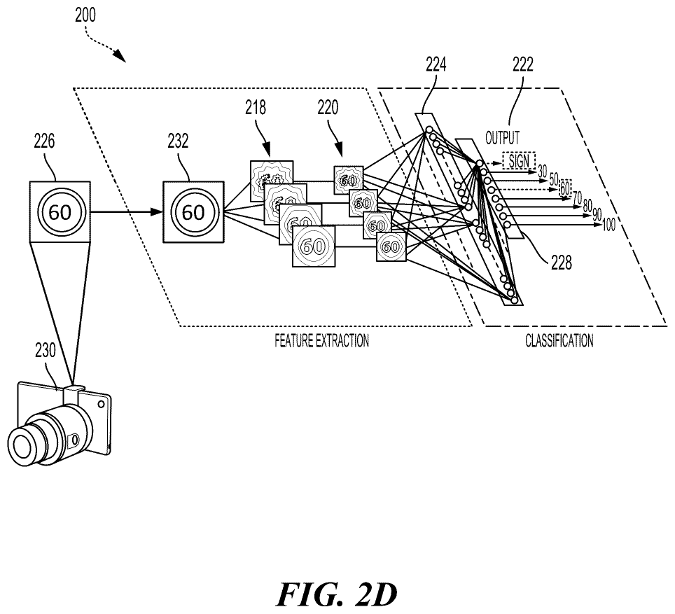

The connections between layers of a neural network may be fully connected or locally connected. FIG. 2A illustrates an example of a fully connected neural network 202. In a fully connected neural network 202, a neuron in a first layer may communicate its output to every neuron in a second layer, so that each neuron in the second layer will receive input from every neuron in the first layer. FIG. 2B illustrates an example of a locally connected neural network 204. In a locally connected neural network 204, a neuron in a first layer may be connected to a limited number of neurons in the second layer. More generally, a locally connected layer of the locally connected neural network 204 may be configured so that each neuron in a layer will have the same or a similar connectivity pattern, but with connection strengths that may have different values (e.g., 210, 212, 214, and 216). The locally connected connectivity pattern may give rise to spatially distinct receptive fields in a higher layer, because the higher layer neurons in a given region may receive inputs that are tuned through training to the properties of a restricted portion of the total input to the network.

One example of a locally connected neural network is a convolutional neural network. FIG. 2C illustrates an example of a convolutional neural network 206. The convolutional neural network 206 may be configured such that the connection strengths associated with the inputs for each neuron in the second layer are shared (e.g., 208). Convolutional neural networks may be well suited to problems in which the spatial location of inputs is meaningful.

One type of convolutional neural network is a deep convolutional network (DCN). FIG. 2D illustrates a detailed example of a DCN 200 designed to recognize visual features from an image 226 input from an image capturing device 230, such as a car-mounted camera. The DCN 200 of the current example may be trained to identify traffic signs and a number provided on the traffic sign. Of course, the DCN 200 may be trained for other tasks, such as identifying lane markings or identifying traffic lights.

The DCN 200 may be trained with supervised learning. During training, the DCN 200 may be presented with an image, such as the image 226 of a speed limit sign, and a forward pass may then be computed to produce an output 222. The DCN 200 may include a feature extraction section and a classification section. Upon receiving the image 226, a convolutional layer 232 may apply convolutional kernels (not shown) to the image 226 to generate a first set of feature maps 218. As an example, the convolutional kernel for the convolutional layer 232 may be a 5×5 kernel that generates 28×28 feature maps. In the present example, because four different convolutional kernels were applied to the image 226 at the convolutional layer 232, four different feature maps are generated in the first set of feature maps 218. The convolutional kernels may also be referred to as filters or convolutional filters.

The first set of feature maps 218 may be subsampled by a max pooling layer (not shown) to generate a second set of feature maps 220. The max pooling layer reduces the size of the first set of feature maps 218. That is, a size of the second set of feature maps 220, such as 14×14, is less than the size of the first set of feature maps 218, such as 28×28. The reduced size provides similar information to a subsequent layer while reducing memory consumption. The second set of feature maps 220 may be further convolved via one or more subsequent convolutional layers (not shown) to generate one or more subsequent sets of feature maps (not shown).

In the example of FIG. 2D, the second set of feature maps 220 is convolved to generate a first feature vector 224. Furthermore, the first feature vector 224 is further convolved to generate a second feature vector 228. Each feature of the second feature vector 228 may include a number that corresponds to a possible feature of the image 226, such as “sign,” “60,” and “100.” A softmax function (not shown) may convert the numbers in the second feature vector 228 to a probability. As such, an output 222 of the DCN 200 is a probability of the image 226 including one or more features.

In the present example, the probabilities in the output 222 for “sign” and “60” are higher than the probabilities of the others of the output 222, such as “30,” “40,” “50,” “70,” “80,” “90,” and “100.” Before training, the output 222 produced by the DCN 200 is likely to be incorrect. Thus, an error may be calculated between the output 222 and a target output. The target output is the ground truth of the image 226 (e.g., “sign” and “60”). The weights of the DCN 200 may then be adjusted so the output 222 of the DCN 200 is more closely aligned with the target output.

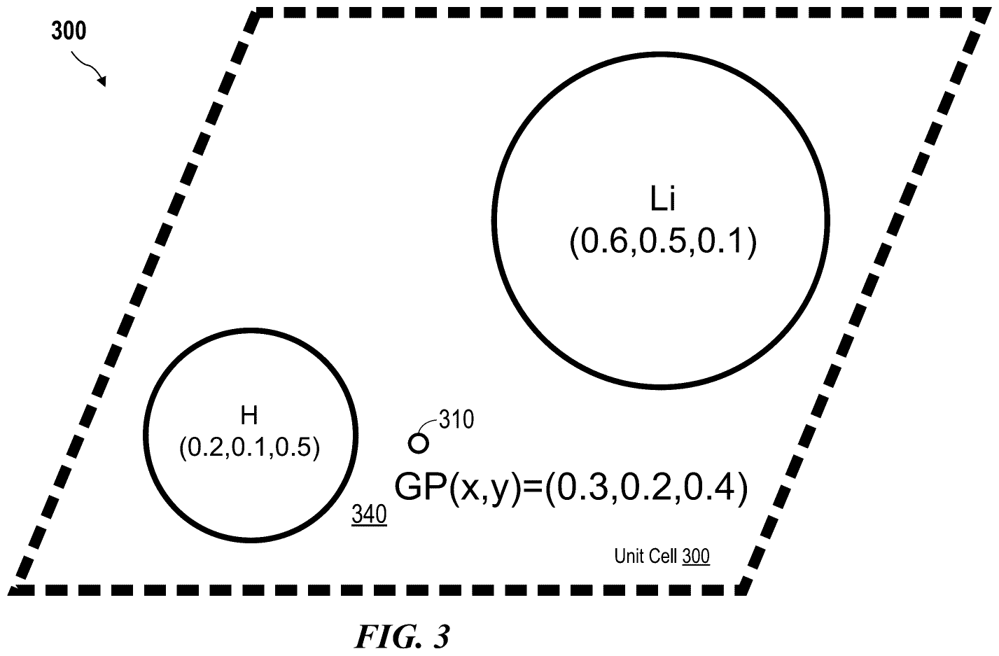

FIG. 3 is a block diagram illustrating a unit cell containing a hydrogen and a lithium atom and their associated three dimensional (3D) embeddings, in accordance with aspects of the present disclosure. As illustrated in FIG. 3, an example point 310 (e.g., 0.3, 0.2, 0.4) is shown within a unit cell 300 based on interpolating of the 3D embeddings of the hydrogen atom 320 (e.g., 0.2, 0.1, 0.5) and the lithium atom 330 (e.g., 0.6, 0.5, 0.1) using a Gaussian process. These aspects of the present disclosure use a Gaussian process to map a crystal structure of the hydrogen atom 320 and the lithium atom 330 to a real space. In this example, the hydrogen atom 320 and the lithium atom 330 of the unit cell 300 serve as training vectors within such a real space to learn properties and/or a high dimensional representation of chemical elements of a material.

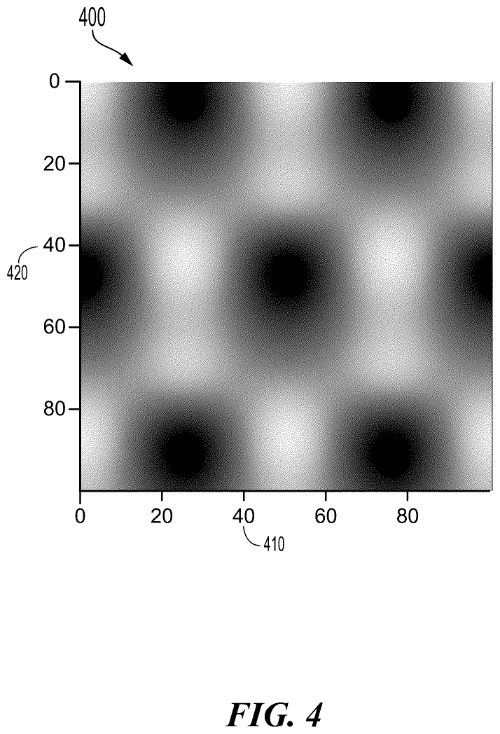

FIG. 4 illustrates a two dimensional (2D) heatmap showing the Gaussian process (GP) interpolations of a single dimension for a 2D hexagonal lattice with two atoms in the unit cell, according to aspects of the present disclosure. In this example, the 2D heatmap 400 includes an X-axis 410 and a Y-axis 420. The 2D heatmap 400 may show the GP interpolations of a single dimension for a 2D hexagonal lattice of the hydrogen atom 320 and the lithium atom 330 in the unit cell 300 of FIG. 3. In some aspects of the present disclosure, the Gaussian process, for any given material, provides a model that represents an atomic structure of the material.

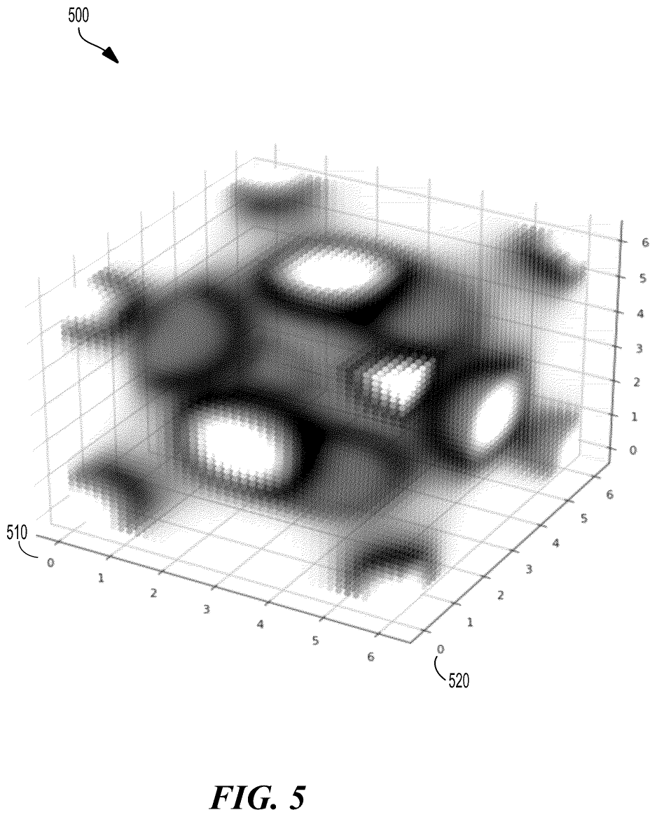

FIG. 5 illustrates a 3D plot 500 showing a bravais lattice of an atom using a face centered cubic (FCC) covariance function, according to aspects of the present disclosure. In this example, the 3D plot 500 includes an X-axis 510, a Y-axis 520, and a Z-axis 530. These aspects of the present disclosure map a unit cell in an atomic structure to periodic boundary conditions, such as the hydrogen atom 320 and the lithium atom 330 in the unit cell 300 of FIG. 3. In this example, the bravais lattices are implemented as periodic covariance functions, e.g., in 3D for simple cubic (SC), face centered cubic (FCC), hexagonal close-packed (HCP), body centered cubic (BCC) as:

sc=(cos(x)+cos(y)+cos(z))/3−1 (1)

fcc=cos(x)*cos(y)*cos(z)−1 (2)

hcp=(cos(2*x)+2*cos(sqrt(3)*y)*cos(x)+cos(2*z))/4−1 (3)

bcc=(cos(x)*cos(y)+cos(x)*cos(z)+cos(z)*cos(y))/3−1 (4)

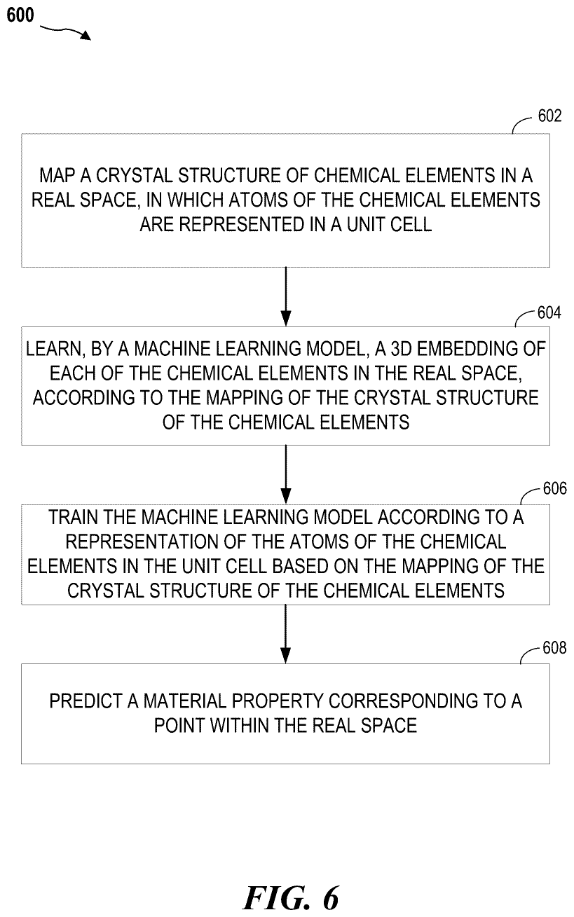

Some aspects of the present disclosure map a unit cell in an atomic structure to periodic boundary conditions, such as the hydrogen atom 320 and the lithium atom 330 in the unit cell 300 of FIG. 3. The periodic boundary conditions are encoded as a Gaussian process. Encoding as a Gaussian process allows one to select any point (e.g., example point 310) in space that includes the unit cell 300 (FIG. 3) and receive a value, such as the coulomb potential. The value at that particular point can be learned. In some aspects of the present disclosure, the training set is a vector for each atom in the unit at their respective positions and identities (X, Y, Z), such as the hydrogen atom 320 and the lithium atom 330 in the unit cell 300 of FIG. 3. The Gaussian process enables acquisition of a value for each embedding at any point in space. Some aspects of the present disclosure learn the embeddings of the elements. These aspects of the present disclosure learn high dimensional representation of chemical elements. These aspects of the present disclosure may predict a property of a material, such as energy density, for example, as further illustrated in FIG. 6.

FIG. 6 is a flow diagram illustrating a method for representing atomic structures as Gaussian processes, according to aspects of the present disclosure. A method 600 begins as block 602, in which a crystal structure of chemical elements is mapped in a real space, in which atoms of the chemical elements are represented in a unit cell. For example, as shown in FIG. 3, a Gaussian process is used to map a crystal structure of the hydrogen atom 320 and the lithium atom 330 to a real space. In this example, the hydrogen atom 320 and the lithium atom 330 of the unit cell 300 serve as training vectors within such a real space to learn properties and/or a high dimensional representation of chemical elements.

At block 604, a machine learning model learns a 3D embedding of each of the chemical elements in the real space, according to the mapping of the crystal structure of the chemical elements. For example, as shown in FIG. 4, the 2D heatmap 400 illustrates Gaussian interpolations of a single dimension for a 2D hexagonal lattice of the hydrogen atom 320 and the lithium atom 330 in the unit cell 300 of FIG. 3. In some aspects of the present disclosure, the Gaussian process, for any given material, provides a model that represents an atomic structure of the material.

At block 606, the machine learning model is trained according to a representation of the atoms of the chemical elements in the unit cell based on the mapping of the crystal structure of the chemical elements. For example, FIG. 5 illustrates mapping of a unit cell in an atomic structure to periodic boundary conditions, such as the hydrogen atom 320 and the lithium atom 330 in the unit cell 300 of FIG. 3. In this example, the periodic boundary conditions are encoded as a Gaussian process. Encoding as a Gaussian process allows one to select any point (e.g., example point 310) in space that includes the unit cell 300 (FIG. 3) and receive a value, such as the coulomb potential. The value at that particular point can be learned.

At block 608, a material property corresponding to a point within the real space is predicted. For example, as shown in FIG. 3, the training set is a vector for each atom in the unit at their respective positions and identities (X, Y, Z), such as the hydrogen atom 320 and the lithium atom 330 in the unit cell 300. The Gaussian process enables acquisition of a value for each embedding at any point in space. Some aspects of the present disclosure learn the embeddings of the elements. These aspects of the present disclosure learn high dimensional representation of chemical elements. These aspects of the present disclosure may predict a property of a material, such as energy density. The method 600 may further include learning properties and/or a high dimensional representation of the chemical elements using the real space.

In some aspects, the method 600 may be performed by the SoC 100 (FIG. 1). That is, each of the elements of the method 600 may, for example, but without limitation, be performed by the SoC 100 or one or more processors (e.g., CPU 102 and/or NPU 108) and/or other components included therein.

The system for accelerating machine learning includes means for dynamically routing inference between the sub-neural networks of a neural network acceleration architecture. In one aspect, the routing means may be the switch device 302 configured to perform the functions recited. In another configuration, the aforementioned means may be any module or any apparatus configured to perform the functions recited by the aforementioned means.

The various operations of methods described above may be performed by any suitable means capable of performing the corresponding functions. The means may include various hardware and/or software component(s) and/or module(s), including, but not limited to, a circuit, an application specific integrated circuit (ASIC), or processor. Generally, where there are operations illustrated in the figures, those operations may have corresponding counterpart means-plus-function components with similar numbering.

As used, the term “determining” encompasses a wide variety of actions. For example, “determining” may include calculating, computing, processing, deriving, investigating, looking up (e.g., looking up in a table, a database or another data structure), ascertaining and the like. Additionally, “determining” may include receiving (e.g., receiving information), accessing (e.g., accessing data in a memory) and the like. Furthermore, “determining” may include resolving, selecting, choosing, establishing, and the like.

As used, a phrase referring to “at least one of” a list of items refers to any combination of those items, including single members. As an example, “at least one of: a, b, or c” is intended to cover: a, b, c, a-b, a-c, b-c, and a-b-c.

The various illustrative logical blocks, modules and circuits described in connection with the present disclosure may be implemented or performed with a general purpose processor, a digital signal processor (DSP), an application specific integrated circuit (ASIC), a field programmable gate array signal (FPGA) or other programmable logic device (PLD), discrete gate or transistor logic, discrete hardware components or any combination thereof designed to perform the functions described. A general purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the present disclosure may be embodied directly in hardware, in a software module executed by a processor, or in a combination of the two. A software module may reside in any form of storage medium that is known in the art. Some examples of storage media that may be used include random access memory (RAM), read-only memory (ROM), flash memory, erasable programmable read-only memory (EPROM), electrically erasable programmable read-only memory (EEPROM), registers, a hard disk, a removable disk, a CD-ROM, and so forth. A software module may comprise a single instruction, or many instructions, and may be distributed over several different code segments, among different programs, and across multiple storage media. A storage medium may be coupled to a processor such that the processor can read information from, and write information to, the storage medium. In the alternative, the storage medium may be integral to the processor.

The methods disclosed, include one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

The functions described may be implemented in hardware, software, firmware, or any combination thereof. If implemented in hardware, an example hardware configuration may comprise a processing system in a device. The processing system may be implemented with a bus architecture. The bus may include any number of interconnecting buses and bridges depending on the specific application of the processing system and the overall design constraints. The bus may link together various circuits including a processor, machine-readable media, and a bus interface. The bus interface may be used to connect a network adapter, among other things, to the processing system via the bus. The network adapter may be used to implement signal processing functions. For certain aspects, a user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further.

The processor may be responsible for managing the bus and general processing, including the execution of software stored on the machine-readable media. The processor may be implemented with one or more general purpose and/or special-purpose processors. Examples include microprocessors, microcontrollers, DSP processors, and other circuitry that can execute software. Software shall be construed broadly to mean instructions, data, or any combination thereof, whether referred to as software, firmware, middleware, microcode, hardware description language, or otherwise. Machine-readable media may include, by way of example, random access memory (RAM), flash memory, read-only memory (ROM), programmable read-only memory (PROM), erasable programmable read-only memory (EPROM), electrically erasable programmable read-only memory (EEPROM), registers, magnetic disks, optical disks, hard drives, or any other suitable storage medium, or any combination thereof. The machine-readable media may be embodied in a computer-program product. The computer-program product may comprise packaging materials.

In a hardware implementation, the machine-readable media may be part of the processing system separate from the processor. However, as those skilled in the art will readily appreciate, the machine-readable media, or any portion thereof, may be external to the processing system. By way of example, the machine-readable media may include a transmission line, a carrier wave modulated by data, and/or a computer product separate from the device, all which may be accessed by the processor through the bus interface. Alternatively, or in addition, the machine-readable media, or any portion thereof, may be integrated into the processor, such as the case may be with cache and/or general register files. Although the various components discussed may be described as having a specific location, such as a local component, they may also be configured in various ways, such as certain components being configured as part of a distributed computing system.

The processing system may be configured as a general purpose processing system with one or more microprocessors providing the processor functionality and external memory providing at least a portion of the machine-readable media, all linked together with other supporting circuitry through an external bus architecture. Alternatively, the processing system may comprise one or more neuromorphic processors for implementing the neuron models and models of neural systems described. As another alternative, the processing system may be implemented with an application specific integrated circuit (ASIC) with the processor, the bus interface, the user interface, supporting circuitry, and at least a portion of the machine-readable media integrated into a single chip, or with one or more field programmable gate arrays (FPGAs), programmable logic devices (PLDs), controllers, state machines, gated logic, discrete hardware components, or any other suitable circuitry, or any combination of circuits that can perform the various functionality described throughout this disclosure. Those skilled in the art will recognize how best to implement the described functionality for the processing system depending on the particular application and the overall design constraints imposed on the overall system.

The machine-readable media may comprise a number of software modules. The software modules include instructions that, when executed by the processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module below, it will be understood that such functionality is implemented by the processor when executing instructions from that software module. Furthermore, it should be appreciated that aspects of the present disclosure result in improvements to the functioning of the processor, computer, machine, or other system implementing such aspects.

If implemented in software, the functions may be stored or transmitted over as one or more instructions or code on a non-transitory computer-readable medium. A non-transitory computer-readable media may include both computer storage media and communication media including any medium that facilitates transfer of a computer program from one place to another. A storage medium may be any available medium that can be accessed by a computer. By way of example, and not limitation, such non-transitory computer-readable media can comprise RAM, ROM, EEPROM, CD-ROM or other optical disk storage, magnetic disk storage, or other magnetic storage devices, or any other medium that can be used to carry or store desired program code in the form of instructions or data structures and that can be accessed by a computer. Additionally, any connection is properly termed a non-transitory computer-readable medium. For example, if the software is transmitted from a web site, server, or other remote source using a coaxial cable, fiber optic cable, twisted pair, digital subscriber line (DSL), or wireless technologies such as infrared (IR), radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk, and Blu-ray® disc, where disks usually reproduce data magnetically, while discs reproduce data optically with lasers. Thus, in some aspects non-transitory computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media.

Thus, certain aspects may comprise a computer program product for performing the operations presented. For example, such a computer program product may comprise a computer-readable medium having instructions stored (and/or encoded) thereon, the instructions being executable by one or more processors to perform the operations described herein. For certain aspects, the computer program product may include packaging material.

Further, it should be appreciated that modules and/or other appropriate means for performing the methods and techniques described herein, may be downloaded and/or otherwise obtained by a user terminal and/or base station as applicable. For example, such a device can be coupled to a server to facilitate the transfer of means for performing the methods described herein. Alternatively, various methods described herein, may be provided via storage means (e.g., RAM, ROM, a physical storage medium such as a compact disc (CD) or floppy disk, etc.), such that a user terminal and/or base station can obtain the various methods upon coupling or providing the storage means to the device. Moreover, any other suitable technique for providing the methods and techniques described herein to a device can be utilized.

It is to be understood that the claims are not limited to the precise configuration and components illustrated above. Various modifications, changes, and variations may be made in the arrangement, operation, and details of the methods and apparatus described above without departing from the scope of the claims.

What is claimed is:

1. A method for representing atomic structures as Gaussian processes, comprising:
mapping a crystal structure of chemical elements in a real space, in which atoms of the chemical elements are represented in a unit cell;
learning, by a machine learning model, a 3D embedding of each of the chemical elements in the real space according to the mapping of the crystal structure of the chemical elements;
training the machine learning model according to a representation of the atoms of the chemical elements in the unit cell based on the mapping of the crystal structure of the chemical elements; and
predicting a material property corresponding to a point within the real space.

2. The method of claim 1, in which mapping comprises mapping the unit cell in the crystal structure to periodic boundary conditions.

3. The method of claim 2, further comprising encoding the periodic boundary conditions as a Gaussian process.

4. The method of claim 1, in which predicting the material property comprises interpolating the 3D embedding of the chemical elements proximate the point in the real space.

5. The method of claim 1, in which the representation of the atoms of the chemical elements in the unit cell based on the mapping of the crystal structure of the chemical elements comprises training vectors for the machine learning model.

6. The method of claim 1, in which predicting comprises estimating an energy density and/or an electron density as the material property of the point.

7. The method of claim 1, further comprising learning properties and/or a high dimensional representation of the chemical elements using the real space.

8. The method of claim 1, in which a training set for the machine learning model comprises a vector for each of the atoms in the unit cell based on respective positions and identities (X, Y, Z).

9. A non-transitory computer-readable medium having program code recorded thereon for representing atomic structures as Gaussian processes, the program code being executed by a processor and comprising:
program code to map a crystal structure of chemical elements in a real space, in which atoms of the chemical elements are represented in a unit cell;
program code to learn, by a machine learning model, a 3D embedding of each of the chemical elements in the real space according to the mapping of the crystal structure of the chemical elements;
program code to train the machine learning model according to a representation of the atoms of the chemical elements in the unit cell based on the mapping of the crystal structure of the chemical elements; and
program code to predict a material property corresponding to a point within the real space.

10. The non-transitory computer-readable medium of claim 9, in which the program code to map comprises program code to map the unit cell in the crystal structure to periodic boundary conditions.

11. The non-transitory computer-readable medium of claim 10, further comprising program code to encode the periodic boundary conditions as a Gaussian process.

12. The non-transitory computer-readable medium of claim 9, in which the program code to predict the material property comprises program code to interpolate the 3D embedding of the chemical elements proximate the point in the real space.

13. The non-transitory computer-readable medium of claim 9, in which the representation of the atoms of the chemical elements in the unit cell being based on the mapping of the crystal structure of the chemical elements comprises training vectors for the machine learning model.

14. The non-transitory computer-readable medium of claim 9, in which the program code to predict comprises program code to estimate an energy density and/or an electron density as the material property of the point.

15. The non-transitory computer-readable medium of claim 9, further comprising program code to learn properties and/or a high dimensional representation of the chemical elements using the real space.

16. The non-transitory computer-readable medium of claim 9, in which a training set for the machine learning model comprises a vector for each of the atoms in the unit cell based on respective positions and identities (X, Y, Z).

17. A system for representing atomic structures as Gaussian processes, the system comprising:
a neural processing unit (NPU);
a memory coupled to the NPU, and instructions stored in the memory and operable, when executed by the NPU, cause the system:
to map a crystal structure of chemical elements in a real space, in which atoms of the chemical elements are represented in a unit cell;
to learn, by a machine learning model, a 3D embedding of each of the chemical elements in the real space according to the mapping of the crystal structure of the chemical elements;
to train the machine learning model according to a representation of the atoms of the chemical elements in the unit cell based on the mapping of the crystal structure of the chemical elements; and
to predict a material property corresponding to a point within the real space.

18. The system of claim 17, in which the instruction to map further causes the system to map the unit cell in the crystal structure to periodic boundary conditions, and to encode the periodic boundary conditions as a Gaussian process.

19. The system of claim 17, in which the instruction further cause the system to learn properties and/or a high dimensional representation of the chemical elements using the real space.

20. The system of claim 17, in which a training set for the machine learning model comprises a vector for each of the atoms in the unit cell based on respective positions and identities (X, Y, Z).
